(19) United States
(12) Patent Application Publication
(10) Pub. No.: US 2025/0276948 A1
(43) Pub. Date: Sep. 4, 2025

(54) 1,1,1,5,5,5-HEXAFLUORO-3-(2,2,2-TRIFLUOROETHOXY)-2-PENTENE AND USES AND PRODUCTION METHOD THEREOF

(71) Applicant: KANTO DENKA KOGYO CO., LTD., Tokyo (JP)

(72) Inventors: Yutaka NAKAMURA, Okayama (JP); Kosuke SANO, Okayama (JP); Natsuho KAZAHAYA, Okayama (JP)

(21) Appl. No.: 18/859,917

(22) PCT Filed: Apr. 27, 2023
(86) PCT No.: PCT/JP2023/016597

§ 371 Date: Oct. 24, 2024

(30) Foreign Application Priority Data
Apr. 28, 2022 (JP) 2022-075050

(51) Int. Cl. C07C 43/17; C07C 41/16; H01M 10/0525; H01M 10/0567; H01M 10/0568; H01M 10/0569

(52) U.S. Cl. CPC C07C 43/17; C07C 41/16; H01M 10/0525; H01M 10/0567; H01M 10/0568; H01M 10/0569; H01M 2300/0037

(57) ABSTRACT

Provided are a novel compound, 1,1,1,5,5,5-hexafluoro-3-(2,2,2-trifluoroethoxy)-2-pentene, and uses thereof and a method for producing this novel compound. According to the present invention, 1,1,1,5,5,5-hexafluoro-3-(2,2,2-trifluoroethoxy)-2-pentene is provided. This novel compound can be produced, for example, by reacting 1,1,1,5,5,5-hexafluoro-3-chloro-2-pentene with 2,2,2-trifluoroethanol in the presence of a base. This novel compound is also useful as an additive in a nonaqueous electrolytic solution for a secondary battery.

### TECHNICAL FIELD

The present invention relates to a novel compound, 1,1,1,5,5,5-hexafluoro-3-(2,2,2-trifluoroethoxy)-2-pentene, and uses of and method for producing this novel compound.

### BACKGROUND ART

Fluoroolefins are used as solvents, cleaning agents, foaming agents, and intermediates for functional materials, for example, and the novel compound of the present invention is also expected to be used for the same or similar uses. For example, in Examples of PTL 1, the self-extinguishing properties of CF₃CH═CHOCH₂CF₃ and CF₃CH═CHOCH₂CF₂CF₂H and the properties of them as electrolyte blends are investigated. However, the above-described novel compounds of the present invention have not been known in the art.

### CITATION LIST

### Patent Literature

PTL 1: International Publication No. WO 2021/064222

### SUMMARY OF INVENTION

### Technical Problem

An object of the present invention is to provide a novel compound, 1,1,1,5,5,5-hexafluoro-3-(2,2,2-trifluoroethoxy)-2-pentene (hereinafter sometimes simply referred to as “3TFEO2HFP”), and uses thereof and a method for producing this novel compound.

### Solution to Problem

The present invention provides the following.
[1] 1,1,1,5,5,5-Hexafluoro-3-(2,2,2-trifluoroethoxy)-2-pentene.
[2] 1,1,1,5,5,5-Hexafluoro-3-(2,2,2-trifluoroethoxy)-2-pentene according to claim 1, represented by the following formula (1).

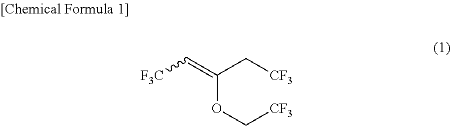 [SMILES: FC(F)(F)C=C(CC(F)(F)F)OCC(F)(F)F]

[3] 1,1,1,5,5,5-Hexafluoro-3-(2,2,2-trifluoroethoxy)-2-pentene according to [1], which is (E)-1,1,1,5,5,5-hexafluoro-3-(2,2,2-trifluoroethoxy)-2-pentene, (Z)-1,1,1,5,5,5-hexafluoro-3-(2,2,2-trifluoroethoxy)-2-pentene, or a combination thereof.
[4] A method for producing 1,1,1,5,5,5-hexafluoro-3-(2,2,2-trifluoroethoxy)-2-pentene, comprising a step of reacting 1,1,1,5,5,5-hexafluoro-3-chloro-2-pentene with 2,2,2-trifluoroethanol in the presence of a base.
[5] The method according to [4], wherein the base is at least one selected from the group consisting of sodium hydroxide, potassium hydroxide and ammonium hydroxide.
[6] The method according to [4] or [5], wherein the base is in the form of an aqueous solution and the reaction is carried out in the presence of a phase-transfer catalyst.
[7] Use of 1,1,1,5,5,5-hexafluoro-3-(2,2,2-trifluoroethoxy)-2-pentene according to any one of [1] to [3] in a nonaqueous electrolytic solution.
[8] The use according to [7], wherein the nonaqueous electrolytic solution is a nonaqueous electrolytic solution for a secondary battery.
[9] A nonaqueous electrolytic solution containing:
an electrolyte;
1,1,1,5,5,5-hexafluoro-3-(2,2,2-trifluoroethoxy)-2-pentene according to any one of [1] to [3]; and
a nonaqueous organic solvent.
[10] The nonaqueous electrolytic solution according to [9], wherein the electrolyte is lithium hexafluorophosphate and the nonaqueous organic solvent is a carbonate solvent.
[11] A secondary battery comprising the nonaqueous electrolytic solution according to [9].
[12] The secondary battery according to [11], wherein the secondary battery is a lithium ion secondary battery.

### Advantageous Effects of Invention

According to the present invention, a novel compound, 1,1,1,5,5,5-hexafluoro-3-(2,2,2-trifluoroethoxy)-2-pentene, and uses thereof and a method for producing this novel compound are provided. It has found that this novel compound is useful particularly as an additive for a nonaqueous electrolytic solution and can be produced using readily available 1,1,1,5,5,5-hexafluoro-3-chloro-2-pentene, 2,2,2-trifluoroethanol and a base such as sodium hydroxide.

### BRIEF DESCRIPTION OF DRAWINGS

FIG. 1 is a cross-sectional diagram of the laminate cell used for the battery evaluation in Examples.

FIG. 2 is a graph showing the progression of capacity retention rate in cycle tests carried out in the battery evaluation in Examples.

### DESCRIPTION OF EMBODIMENTS

### Effects

As shown in formula (1), the novel compound of the present invention has a special chemical structure in which a trifluoromethyl group and two groups each having a trifluoromethyl group at the terminal (2,2,2-trifluoroethyl group and 2,2,2-trifluoroethoxy group) are attached at three of the four positions on the C═C double bond, on which substitution can be made. Since this compound has a relatively high molecular weight of 276, it has a boiling point of 125 to 130° C. at atmospheric pressure and therefore is liquid at ordinary temperatures and pressures. This compound is self-extinguishing due to the presence of nine fluorine atoms in its molecule. Two of the trifluoromethyl groups extending outward the molecule are linked to the C═C double bond, which is rigid, via a methylene group (—CH₂—), and accordingly, the two have a relatively high degree of freedom so that the external attack of a reaction reagent to the C═C double bond is moderately limited. The 2,2,2-trifluoroethoxy group attached to the C═C double bond is affected by the electron-withdrawing property of the terminal trifluoromethyl group, and it gives a moderate electron-withdrawing property to the C═C double bond due to the intervention of a methylene group, as compared to the trifluoromethoxy group. Therefore, the reactivity of the C═C double bond is moderately limited. Due to such a chemical structure, the novel compound of the present invention is characterized in that it is liquid at ordinary temperatures and pressures, and self-extinguishing and can be used in a chemically stable state, and also characterized in that after use, it can be recovered as low-molecular compounds as a result of cleavage of the C═C double bond and the ether bond. Based on these characteristics, the novel compound of the present invention is believed to be useful as a solvent, a cleaning agent, a foaming agent, and an intermediate for functional materials, for example. As described later in Examples, the present inventors have newly found that the novel compound of the present invention can form a good film on the positive electrode and negative electrode interfaces of a secondary battery, thereby providing excellent battery properties. A nonaqueous organic solvent is generally used for an electrolytic solution for a secondary battery, and lithium hexafluorophosphate (LiPF₆) is added thereto as an electrolyte. The novel compound of the present invention has high affinity with a fluorine compound due to its three trifluoromethyl groups, whereas it also has a high affinity with a nonaqueous organic solvent and can be uniformly mixed in an electrolytic solution, due to its carbon chain as the basic skeleton. In addition, this compound can be stably present in an electrically polarized state due to the conjugation of between the C═C double bond and the ether bond, which are located in the center of its molecule, and accordingly, it has a high affinity with the positive electrode and negative electrode interfaces and can be stably present between the positive electrode and negative electrode. This compound is believed to be useful as an additive for an electrolytic solution for a secondary battery due to such characteristics based on its chemical structure.

### [Method for Producing 3TFEO2HFP]

The novel compound of the present invention, 1,1,1,5,5,5-hexafluoro-3-(2,2,2-trifluoroethoxy)-2-pentene can be produced by a step of reacting 1,1,1,5,5,5-hexafluoro-3-chloro-2-pentene with 2,2,2-trifluoroethanol in the presence of a base.

The starting materials for production, 1,1,1,5,5,5-hexafluoro-3-chloro-2-pentene and 2,2,2-trifluoroethanol, are both known substances, and they can be easily produced by any known method or is easily available as a reagent. Trifluoroethanol may be used in a slight excess amount per mole of 1,1,1,5,5,5-hexafluoro-3-chloro-2-pentene, and trifluoroethanol is used preferably in an amount of 1 to 1.5 moles, more preferably 1 to 1.3 moles, and most preferably 1 to 1.2 moles, per mole of 1,1,1,5,5,5-hexafluoro-3-chloro-2-pentene in the reaction.

Examples of the base include inorganic bases such as alkali metal hydroxides (such as sodium hydroxide and potassium hydroxide); hydroxides such as ammonium hydroxide; hydrogen carbonates and carbonates such as alkali metal hydrogen carbonates and carbonates (such as sodium hydrogen carbonate and sodium carbonate); and metal hydrides such as alkali metal hydrides (such as sodium hydride and potassium hydride); and also organic bases such as alkylamines (such as triethylamine, tetramethylethylenediamine and N,N-diisopropylamine), 1-azabicyclo[2.2.2]octane, 1,4-diazabicyclo[2.2.2]octane (abbreviated name: DABCO), 1,8-diazabicyclo[5.4.0]undec-7-ene (abbreviated name: DBU), pyridine, 4-(dimethylamino)pyridine, pyrrolidine, piperidine and morpholine. The base is preferably sodium hydroxide, potassium hydroxide and ammonium hydroxide, and particularly preferably sodium hydroxide (caustic soda). The base may be used in an excess amount per mole of 1,1,1,5,5,5-hexafluoro-3-chloro-2-pentene as a starting material, and the base is used preferably in an amount of 1 to 3 moles, more preferably 1 to 2 moles, and most preferably 1 to 1.5 moles, per mole of 1,1,1,5,5,5-hexafluoro-3-chloro-2-pentene in the reaction. When the base is used in the form of an aqueous solution, the base concentration of the aqueous solution is preferably 5 to 30% by weight, more preferably 5 to 20% by weight, and most preferably 10 to 15% by weight, based on the total amount of the aqueous solution.

A phase-transfer catalyst can be present in the reaction solution. When the base to be used in the present invention is in the form of an aqueous solution, the active species of the reaction are present in the aqueous phase. On the other hand, since the starting materials, 1,1,1,5,5,5-hexafluoro-3-chloro-2-pentene and 2,2,2-trifluoroethanol are lipophilic, they are present in the oil phase and do not dissolve in the aqueous phase. Therefore, if the phase-transfer catalyst is used, the active species in the aqueous phase can be transferred to the oil phase to promote the reaction. Examples of the phase-transfer catalyst include quaternary ammonium salts such as tetrabutylammonium bromide and tetrabutylammonium chloride; quaternary phosphonium salts such as tetrabutylphosphonium chloride and tetrabutylphosphonium bromide; and crown ethers such as 12-crown-4, 15-crown-5 and 18-crown-6. The phase-transfer catalyst is preferably tetrabutylammonium bromide and tetrabutylammonium chloride, and particularly preferably tetrabutylammonium bromide. The phase-transfer catalyst is used in an amount of 0.005 to 0.1 moles, more preferably 0.01 to 0.05 moles, and most preferably 0.01 to 0.02 moles, per mole of 1,1,1,5,5,5-hexafluoro-3-chloro-2-pentene as a starting material.

When using a solvent in addition to water, it is possible to use water-miscible solvents such as amides such as dimethylformamide (DMF), dimethyl sulfoxide (DMSO) and N-methylpyrrolidone (NMP); lower alcohols such as methanol and ethanol; ethers such as tetrahydrofuran (THF) and 1,4-dioxane; and acetonitrile. Even when using a water-immiscible organic solvent and water, the active species of the reaction can be present in both the water phase and the oil phase (organic solvent phase) by using the above-described phase-transfer catalyst. Examples of such an organic solvent that can be used include ether-based solvents such as diethyl ether; halogenated hydrocarbon-based solvents such as dichloromethane; aromatic hydrocarbon-based solvents such as benzene, toluene and xylene; and aliphatic hydrocarbon-based solvents such as pentane, hexane and octane. Preferred examples of the solvent are water, DMF and acetonitrile.

The reaction temperature is preferably −40° C. to 120° C., more preferably −20° C. to 60° C., even more preferably −10° C. to 40° C., and most preferably 0° C. to 25° C. The reaction pressure may be an ordinary pressure, and a glass reactor can be used.

### [Uses of 3TFEO2HFP]

As described above, the novel compound of the present invention is characterized, by its chemical structure, in that it is liquid at ordinary temperatures and pressures, and self-extinguishing and can be used in a chemically stable state, and also characterized in that after use, it can be recovered as low-molecular compounds as a result of cleavage of the C═C double bond and the ether bond. Based on these characteristics, the novel compound of the present invention is believed to be useful as a solvent, a cleaning agent, a foaming agent, and an intermediate for functional materials, for example.

In addition, the present inventors have newly found that the novel compound of the present invention can form a good film on the positive electrode and negative electrode interfaces of a secondary battery, thereby providing excellent battery properties, as described later in Examples. When the novel compound of the present invention is used as an additive for an electrolytic solution for a secondary battery, it is added to the electrolytic solution in an amount of 0.005 to 10% by weight, more preferably 0.1 to 7.5% by weight, and most preferably 1 to 5% by weight, based on 100% by weight of the total amount of electrolytic solution.

Examples of the preferred composition of the electrolytic solution having the novel compound of the present invention added thereto include the following composition.

### (Range of Composition of Electrolytic Solution)

(1) Nonaqueous organic solvent: balance

Examples of the nonaqueous organic solvent include, but not particularly limited to, a carbonate solvent such as ethylene carbonate or ethyl methyl carbonate, a chain carbonate ester, a phosphate ester, a cyclic ether, a chain ether, a lactone compound, a chain ester, a nitrile compound, an amide compound and a sulfone compound. Among these organic solvents, a carbonate solvent is preferred in view of common use as an organic solvent for a lithium secondary battery.
(2) Electrolyte: preferably 0.1 to 2 mol/L, more preferably 0.15 to 1.8 mol/L, and most preferably 0.3 to 1.2 mol/L with regard to the volume of the solvent.

Examples of the electrolyte include, but not particularly limited to, fluorine-based electrolytes such as lithium hexafluorophosphate (LiPF₆), lithium borofluoride (LiBF₄), lithium trifluoromethanesulfonate (LiCF₃SO₃), LiBF₃CF₃, LiBF₃C₂F₅, LiC₂F₅SO₃, LiC₃F₇SO₃, LiC₄F₉SO₃, LiN(SO₂F)₂, LiN(CF₃SO₂)₂, LiN(C₂F₅SO₂)₂, LiN(C₂F₅SO₂)₂, LiN(CF₃SO₂)(CF₃CO), LiN(CF₃SO₂)(C₂F₅SO₂) and LiC(CF₃SO₂)₃. The electrolytes can be used singly or in combination of two or more thereof. Among these fluorine-based electrolyte, LiPF₆ is preferred in view of safety and stability of a nonaqueous electrolytic solution as well as improvement in electrical conductivity and cycle characteristics of a nonaqueous electrolytic solution.
(3) Novel compound of the present invention: it is added to the electrolytic solution, preferably in amount of 0.005 to 10% by weight, more preferably 0.1 to 7.5% by weight, and most preferably 1 to 5% by weight, based on 100% by weight of the total amount of electrolytic solution.
(4) Other optional additive components (such as fluoroethyl carbonate (FEC), vinylene carbonate (VC) and lithium difluorophosphate): the amounts thereof added are smaller than those of the solvent and the electrolyte, and are used as additives expected to exhibit the effect of improving battery performance, such as film formation. These are added to the electrolytic solution, preferably in amount of 0.005 to 10% by weight, more preferably 0.1 to 7.5% by weight, and most preferably 1 to 5% by weight, based on 100% by weight of the total amount of electrolytic solution.

### EXAMPLES

### Production Example 1

Into a 500 mL three-neck glass flask were introduced 89.3 g of 3-chloro-1,1,1,5,5,5-hexafluoro-2-pentene, 50.4 g of 2,2,2-trifluoroethanol and 1.62 g of tetrabutylammonium bromide, followed by dropwise addition of 268 g of 12.5% aqueous caustic soda solution over 25 minutes. After stirring at room temperature (25° C.) for 10 minutes, the production of 1,1,1,5,5,5-hexafluoro-3-(2,2,2-trifluoroethoxy)-2-pentene was confirmed by NMR. After washing with water, it was subjected to purification by distillation to obtain 76.7 g (yield: 66%) of 1,1,1,5,5,5-hexafluoro-3-(2,2,2-trifluoroethoxy)-2-pentene with a GC purity of 99% or more. Analysis of the product shows that it was obtained at the isomer ratio (E)/(Z) of 30/1.

### Production Example 2

Into a 500 mL three-neck glass flask were introduced 100 g of 3-chloro-1,1,1,5,5,5-hexafluoro-2-pentene, 47.1 g of 2,2,2-trifluoroethanol and 1.82 g of tetrabutylammonium bromide, followed by dropwise addition of 151 g of 12.5% aqueous caustic soda solution. After stirring at room temperature (25° C.) for 12 hours, the production of 1,1,1,5,5,5-hexafluoro-3-(2,2,2-trifluoroethoxy)-2-pentene was confirmed by NMR. After washing with water, it was subjected to purification by distillation to obtain 117 g (yield: 90%) of 1,1,1,5,5,5-hexafluoro-3-(2,2,2-trifluoroethoxy)-2-pentene with a GC purity of 99% or more. Analysis of the product shows that it was obtained at the isomer ratio (E)/(Z) of 30/1. The boiling point of the product (mixture of isomers) was 73 to 76° C. at 180 to 190 hPa and 125 to 130° C. at atmospheric pressure.

The NMR and GC-MS data for (E)-1,1,1,5,5,5-hexafluoro-3-(2,2,2-trifluoroethoxy)-2-pentene are shown below.

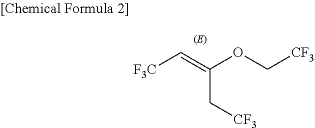 [SMILES: FC(F)(F)/C=C(\CC(F)(F)F)OCC(F)(F)F]

¹H-NMR (400 MHZ); δ 3.25 (q, JHF=9.6 Hz, 2H), 4.10 (q, JHF=7.2 Hz, 2H), 5.00 (q, JHF=7.6 Hz, 1H).

¹⁹F-NMR (376 MHz); δ −63.9-−64.1 (m, 3F), −55.0-−55.1 (m, 3F), −74.2-−74.3 (m, 3F) GC-MS m/z (%): 276 (CF₃CH═C(CH₂CF₃)OCH₂CF₃), 207 (CF₃CH═C(CH₂)OCH₂CF₃), 193 (CF₃CH═COCH₂CF₃), 83 (CH₂CF₃).

The NMR and GC-MS data for (Z)-1,1,1,5,5,5-hexafluoro-3-(2,2,2-trifluoroethoxy)-2-pentene are shown below.

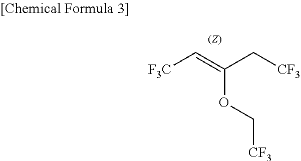 [SMILES: FC(F)(F)/C=C(/CC(F)(F)F)OCC(F)(F)F]

¹H-NMR (400 MHZ); δ 3.02 (q, J_{HF}=9.6 Hz, 2H), 4.26 (q, J_{HF}=7.6 Hz, 2H), 5.23 (q, J_{HF}=8.0 Hz, 1H).

¹⁹F-NMR (376 Mhz); δ −64.8-−65.0 (m, 3F), −57.5-−57.6 (m, 3F), −75.1-−75.2 (m, 3F) GC-MS m/z (%): 276 (CF₃CH═C(CH₂CF₃)OCH₂CF₃), 207 (CF₃CH═C(CH₂)OCH₂CF₃), 193 (CF₃CH═COCH₂CF₃), 83 (CH₂CF₃).

### [Evaluation of Battery]

In order to clarify that the novel compound, 1,1,1,5,5,5-hexafluoro-3-(2,2,2-trifluoroethoxy)-2-pentene represented by formula (1) obtained in each of the above Production Examples has the effect of forming a good film, a nonaqueous electrolytic solution secondary battery comprising a nonaqueous electrolytic solution containing 1,1,1,5,5,5-hexafluoro-3-(2,2,2-trifluoroethoxy)-2-pentene was subjected to an evaluation test. In addition, the same compound as in PTL 1 (CF₃CH═CHOCH₂CF₃) was synthesized and subjected to a battery evaluation, to compare the effect of the addition of the novel compound with that of the compound described in Examples of PTL 1.

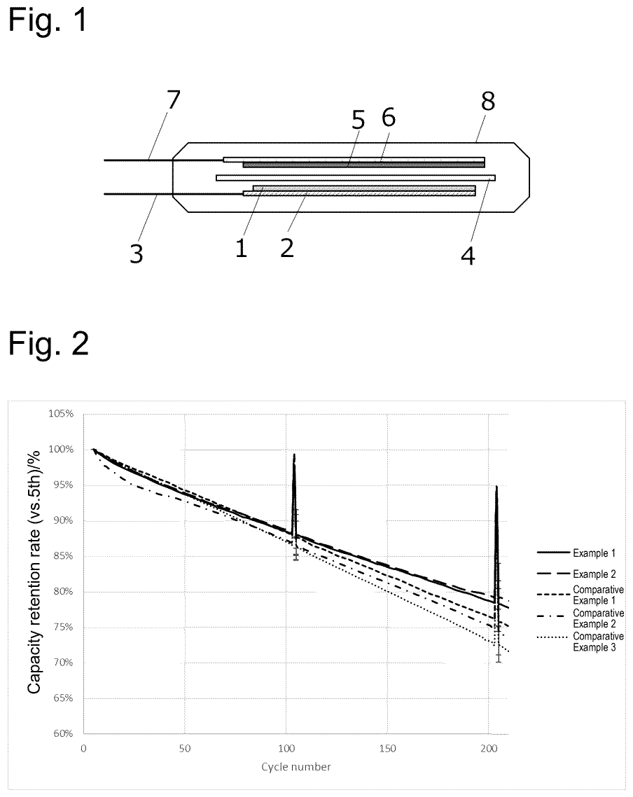

In the present evaluation test, the nonaqueous electrolytic solution secondary battery of a laminate cell as shown in FIG. 1 was fabricated by using an electrolytic solution containing 1,1,1,5,5,5-hexafluoro-3-(2,2,2-trifluoroethoxy)-2-pentene obtained in each of Production Example 1 or Production Example 2.

### <Fabrication Procedure>

### [Structure of Battery Used]

The cross-section structure of the laminate cell used in the present evaluation test is shown in FIG. 1. In this laminate cell, positive electrode material 1 coats positive electrode current collector 2; positive electrode tab 3 extending from positive electrode current collector 2 can be electrically connected to a wiring from a measuring device with a clip; negative electrode material 5 coats on negative electrode current collector 6; negative electrode tab 7 extending from negative electrode current collector 6 can be electrically connected to a wiring from the measuring device with a clip; and separator 4 is placed between positive electrode material 1 and negative electrode material 5. Each member has a flat rectangular shape. The members are arranged in layers in the following order: positive electrode current collector 2, positive electrode material 1, separator 4, negative electrode material 5 and negative electrode current collector 6, and are housed in laminate sheath 8.

### [Battery Members Used]

The positive electrode to be used was a lithium nickel cobalt manganese oxide (NCM111) electrode applied onto an aluminum current collector, and the negative electrode to be used was an artificial graphite electrode applied onto a nickel current collector. The thickness of the positive electrode material (coating area in the electrode) was 53 μm, and the thickness of the negative electrode material was about 56 μm. A polypropylene microporous membrane (manufactured by Celgard; the trade name “Celgard #2400”) was cut to 5 cm×6.5 cm and used as the separator. The laminate sheath to be used was obtained by folding a laminate with a size of 11 cm×20 cm in half, and a laminate cell with the configuration shown in FIG. 1 was fabricated.

### [Assembling Battery]

In the above laminate cell, NCM111 was used for the positive electrode and artificial graphite was used for the negative electrode, each of which was used after being cut to a specified size (positive electrode: 4 cm×5 cm, negative electrode: 4.5 cm×5 cm). A tab for clamping an alligator clip was welded to each electrode, and a separator (5 cm×6.5 cm) was sandwiched between the positive electrode and the negative electrode, which was in turn placed in the laminate cell and dried for 1 hour at 100° C. Thereafter, 0.5 mL of the electrolytic solution containing the compound of the present invention represented by formula (1) synthesized in Production Example 1 was poured thereinto in an Ar glove box, and the laminate cell was sealed using a vacuum sealer.

### Example 1

Ethylene carbonate (EC) and ethyl methyl carbonate (EMC) were mixed at a volume ratio of EC:EMC=3:7 to obtain a nonaqueous solvent, and in the nonaqueous solvent, lithium hexafluorophosphate LiPF₆ as an electrolyte was dissolved to a content of 1 mol/L to obtain a solution. To the solution, the compound of the present invention represented by formula (I) was added in an amount of 1% by weight based on the weight of the solution to prepare a nonaqueous electrolytic solution. Then, a nonaqueous electrolytic solution secondary battery of a laminate cell as shown in FIG. 1 was fabricated.

### Example 2

Ethylene carbonate (EC) and ethyl methyl carbonate (EMC) were mixed at a volume ratio of EC:EMC=3:7 to obtain a nonaqueous solvent, and in the nonaqueous solvent, lithium hexafluorophosphate LiPF₆ as an electrolyte was dissolved to a content of 1 mol/L to obtain a solution. To the solution, the compound of the present invention represented by formula (I) was added in an amount of 5% by weight based on the weight of the solution to prepare a nonaqueous electrolytic solution. Then, a nonaqueous electrolytic solution secondary battery of a laminate cell as shown in FIG. 1 was fabricated.

### Comparative Example 1

Ethylene carbonate (EC) and ethyl methyl carbonate (EMC) were mixed at a volume ratio of EC:EMC=3:7 to obtain a nonaqueous solvent, and in the nonaqueous solvent, lithium hexafluorophosphate LiPF₆ as an electrolyte was dissolved to a content of 1 mol/L.

### Comparative Example 2

Ethylene carbonate (EC) and ethyl methyl carbonate (EMC) were mixed at a volume ratio of EC:EMC=3:7 to obtain a nonaqueous solvent, and in the nonaqueous solvent, lithium hexafluorophosphate LiPF₆ as an electrolyte was dissolved to a content of 1 mol/L to obtain a solution. To the solution, CF₃CH═CHOCH₂CF₃ was added in an amount of 1% by weight based on the weight of the solution to prepare a nonaqueous electrolytic solution. Then, a nonaqueous electrolytic solution secondary battery of a laminate cell as shown in FIG. 1 was fabricated.

### Comparative Example 3

Ethylene carbonate (EC) and ethyl methyl carbonate (EMC) were mixed at a volume ratio of EC:EMC=3:7 to obtain a nonaqueous solvent, and in the nonaqueous solvent, lithium hexafluorophosphate LiPF₆ as an electrolyte was dissolved to a content of 1 mol/L to obtain a solution. To the solution, CF₃CH═CHOCH₂CF₃ was added in an amount of 5% by weight based on the weight of the solution. Then, a nonaqueous electrolytic solution secondary battery of a laminate cell as shown in FIG. 1 was fabricated.

### [Measurement Method]

In order to vent the gas initially generated, the nonaqueous electrolytic solution secondary battery fabricated in each of Examples 1 and 2 and Comparative Examples 1 to 3 was subjected to constant-current charging at 0.2 C at a temperature of 60° C. to reach 30% of the theoretical capacity, and then stored for 15 hours. (Aging, 1^{st} charging)

Thereafter, each of the above-described aged nonaqueous electrolytic solution secondary batteries fabricated in each of Examples 1 and 2 and Comparative Examples 1 to 3 was placed in an Ar glove box. The edge of the laminate cell was cut, and gas was vented. The laminate cell was sealed again in a vacuum state with a laminator. (Gas venting)

Next, each of the nonaqueous electrolytic solution secondary batteries of Examples 1 and 2 and Comparative Examples 1 to 3 was subjected to constant-current charging at 0.2 C at a temperature of 25° C. to reach 4.2 V, and further subjected to constant-voltage charging at a charging termination C current of 0.02 C until the current value reached 1/50 of the theoretical capacity corresponding to the weight of the active material, and then subjected to discharging at a constant current of 0.2 C to reach 2.75 V. (2^{nd} and 3^{rd} charging/discharging)

Next, each of the nonaqueous electrolytic solution secondary batteries of Examples 1 and 2 and Comparative Examples 1 to 3 was subjected to constant-current charging at 1 C at a temperature of 25° C. to reach 4.2 V, and further subjected to constant-voltage charging at a charging termination C current of 0.1 C until the current value reached 1/10 of the theoretical capacity corresponding to the weight of the active material, and then subjected to discharging at a constant current of 1 C to reach 50% of the theoretical capacity. (4^{th} charging/discharging)

Thereafter, each of the nonaqueous electrolytic solution secondary batteries of Examples 1 and 2 and Comparative Examples 1 to 3 was subjected to constant-current charging at 1 C at a temperature of 45° C. to reach 4.2 V, and further subjected to constant-voltage charging at a charging termination C current of 0.1 C until the current value reached 1/10 of the theoretical capacity corresponding to the weight of the active material, and then subjected to discharging at a constant current of 1 C to reach 2.75 V. (5^{th}˜charging/discharging)

Every 100 cycles, the second battery was subjected to constant-current charging at 0.2 C to reach 4.2 V, further subjected to constant-voltage charging at a charging termination C current of 0.1 C until the current value reached 1/10 of the theoretical capacity corresponding to the weight of the active material, and then subjected to discharging at a constant current of 1 C to reach 2.75 V.

Based on the first (5^{th}) discharge capacity D₅ and the x^{th} discharge capacity Dₓ (x=105, 205) measured at a temperature of 45° C. and at 1C as described above, the capacity retention rate (%) after the x^{th} cycle of each of the nonaqueous electrolytic solution secondary batteries of Examples 1 and 2 and Comparative Examples 1 to 3 was calculated using the following equation. The results are shown in Table 1.

$$Capacity retention rate ( \% ) = ( {D}_{x} / {D}_{5} ) \times 100$$

TABLE 1

| | | | Capacity retention | Capacity retention |
|---|---|---|---|---|
| | | Amount | rate (%) at 105^{th} | rate (%) at 205^{th} |
| | Additive | added | cycle | cycle |
| Example 1 | CF₃CH═C(OCH₂CF₃)CH₂CF₃ | 1 wt % | 88.0% | 78.3% |
| Example 2 | CF₃CH═C(OCH₂CF₃)CH₂CF₃ | 5 wt % | 88.0% | 79.2% |
| Comparative | None | — | 87.6% | 75.8% |
| Example 1 | | | | |
| Comparative | CF₃CH═CHOCH₂CF₃ | 1 wt % | 86.5% | 74.3% |
| Example 2 | | | | |
| Comparative | CF₃CH═CHOCH₂CF₃ | 5 wt % | 86.3% | 72.6% |
| Example 3 | | | | |

Table 1 shows that Examples 1 and 2 had a higher capacity retention rate at the 105^{th} cycle than Comparative Examples 1 to 3 and were excellent in cycle characteristics. At the 205^{th} cycle, further differences in capacity retention rate occurred between Examples and Comparative Examples, and the highest capacity retention rate was exhibited in Example 2. It is concluded that 1,1,1,5,5,5-hexafluoro-3-(2,2,2-trifluoroethoxy)-2-pentene of the present invention represented by formula (1) can form a good film on the positive electrode and negative electrode interfaces, thereby providing excellent battery characteristics.

1. 1,1,1,5,5,5-Hexafluoro-3-(2,2,2-trifluoroethoxy)-2-pentene.

2. 1,1,1,5,5,5-Hexafluoro-3-(2,2,2-trifluoroethoxy)-2-pentene according to claim 1, represented by the following formula (1).

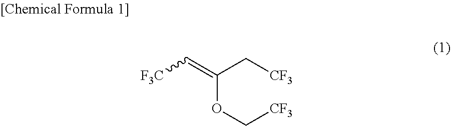 [SMILES: FC(F)(F)C=C(CC(F)(F)F)OCC(F)(F)F]

3. 1,1,1,5,5,5-Hexafluoro-3-(2,2,2-trifluoroethoxy)-2-pentene according to claim 1, which is (E)-1,1,1,5,5,5-hexafluoro-3-(2,2,2-trifluoroethoxy)-2-pentene, (Z)-1,1,1,5,5,5-hexafluoro-3-(2,2,2-trifluoroethoxy)-2-pentene, or a combination thereof.

4. A method for producing 1,1,1,5,5,5-hexafluoro-3-(2,2,2-trifluoroethoxy)-2-pentene, comprising a step of reacting 1,1,1,5,5,5-hexafluoro-3-chloro-2-pentene with 2,2,2-trifluoroethanol in the presence of a base.

5. The method according to claim 4, wherein the base is at least one selected from the group consisting of sodium hydroxide, potassium hydroxide and ammonium hydroxide.

6. The method according to claim 4, wherein the base is in the form of an aqueous solution and the reaction is carried out in the presence of a phase-transfer catalyst.

7. Use of 1,1,1,5,5,5-hexafluoro-3-(2,2,2-trifluoroethoxy)-2-pentene according to claim 1 in a nonaqueous electrolytic solution.

8. The use according to claim 7, wherein the nonaqueous electrolytic solution is a nonaqueous electrolytic solution for a secondary battery.

9. A nonaqueous electrolytic solution comprising:
an electrolyte;
1,1,1,5,5,5-hexafluoro-3-(2,2,2-trifluoroethoxy)-2-pentene according to claim 1; and
a nonaqueous organic solvent.

10. The nonaqueous electrolytic solution according to claim 9, wherein the electrolyte is lithium hexafluorophosphate and the nonaqueous organic solvent is a carbonate solvent.

11. A secondary battery comprising the nonaqueous electrolytic solution according to claim 9.

12. The secondary battery according to claim 11, wherein the secondary battery is a lithium ion secondary battery.
